(19) United States
(12) Patent Application Publication
(10) Pub. No.: US 2007/0225805 A1
(43) Pub. Date: Sep. 27, 2007

(54) Ligament Fixation Using Graft Harness/Bolt Assembly

(76) Inventor: Reinhold Schmieding, Naples, FL (US)

Correspondence Address: DICKSTEIN SHAPIRO LLP, 1825 EYE STREET NW, Washington, DC, 20006-5403, US

(21) Appl. No.: 11/687,695

(22) Filed: Mar. 19, 2007

Related U.S. Application Data
(60) Provisional application No. 60/783,867, filed on Mar. 21, 2006.

(51) Int. Cl. A61F 2/08

(52) U.S. Cl. 623/13.14

(57) ABSTRACT

An apparatus and method for fixation of a soft tissue ligament graft in a bone tunnel using a graft harness/bolt assembly. The graft harness/bolt assembly includes a harness for supporting a looped graft, and a bolt configured to be received in, and securely engage, the harness, the inserted bolt spreading the legs of the harness to engage the walls of a bone tunnel. The bolt includes a transverse hole for receiving a suture strand, which is used to draw and fixate the graft harness assembly into the bone tunnel.

This application claims priority to U.S. Provisional Application No. 60/783,867, filed on Mar. 21, 2006, the entire contents of which are hereby incorporated by reference.

### FIELD OF THE INVENTION

The present invention relates to apparatus and methods for ligament fixation and, more particularly, to techniques using a graft harness/bolt assembly for fixation of soft tissue ligament grafts in a bone tunnel.

### BACKGROUND OF THE INVENTION

When soft tissues, such as tendons or ligaments, tear or detach from bone, it is often necessary to reconnect the structures surgically. Cruciate ligament reconstruction is routinely performed by creating femoral and tibial tunnels into which ligament grafts are secured. Various types of graft are used to replace the native cruciate ligament. Biomechanical studies have shown that an anatomic double-bundle cruciate ligament reconstruction is superior in restoring normal knee laxity compared with conventional single-bundle isometric reconstructions. Improved methods for installing and securing double-bundle cruciate ligament grafts could enhance reconstruction results.

### SUMMARY OF THE INVENTION

The present invention provides ligament graft fixation using a graft harness/bolt assembly comprising a graft harness and a bolt that is securely received within the harness, and spreads the legs of the harness to engage the wall of a bone tunnel. The harness is provided with a groove at its distal end which receives and supports a looped ligament graft. At least a portion of the looped ligament graft may be securely attached to the harness by suturing.

The bolt is provided with grooves that securely engage corresponding protuberances of the harness, to allow the bolt to lock on the harness. A suture passageway formed through the bolt, and preferably transverse to the groove of the harness, accepts a length of suture which is used to draw and fixate the graft harness/bolt assembly into a bone tunnel, such as a femoral socket or tunnel (“femoral opening”). The graft harness/bolt assembly provides ligament graft fixation within the femoral opening, with a predetermined graft bundle orientation.

Other features and advantages of the present invention will become apparent from the following description of the invention which refers to the accompanying drawings.

### BRIEF DESCRIPTION OF THE DRAWINGS

FIG. 1 illustrates a graft harness according to an embodiment of the present invention;

FIG. 2 illustrates a bolt used in conjunction with the graft harness of FIG. 1 and according to an embodiment of the present invention;

FIG. 3 illustrates a top view of the bolt of FIG. 2;

FIG. 4 illustrates a side view of the bolt of FIG. 2;

FIG. 5 illustrates a lateral view of the bolt of FIG. 2;

FIG. 6 illustrates a bottom view of the graft harness of FIG. 1;

FIG. 7 illustrates a front view of the graft harness of FIG. 1;

FIG. 8 illustrates another front view of the graft harness of FIG. 1;

FIG. 9 illustrates a top view of the graft harness of FIG. 1; and

FIG. 10 illustrates a partially cut-away side view of a knee showing fixation of the harness/bolt graft assembly of the present invention in the knee.

### DETAILED DESCRIPTION OF THE INVENTION

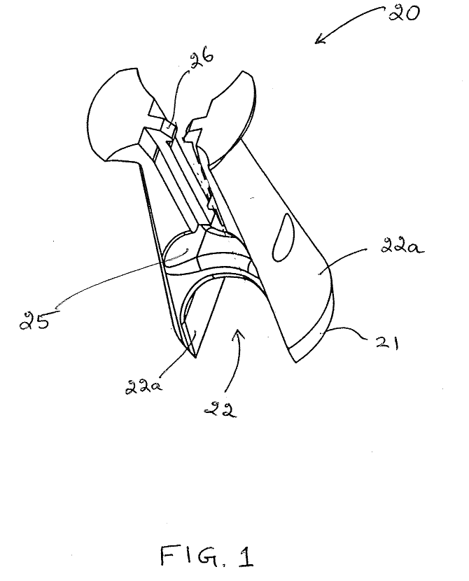

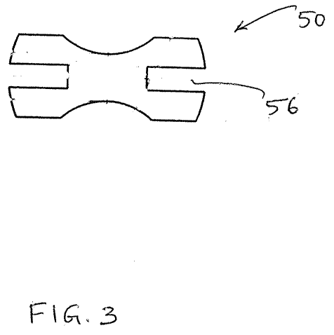

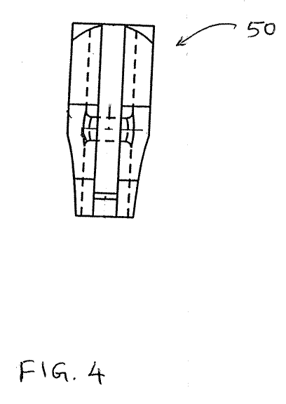

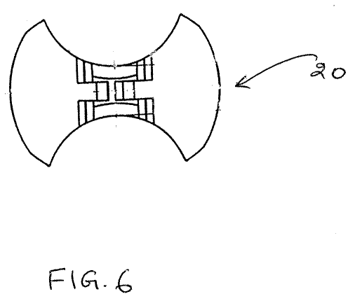

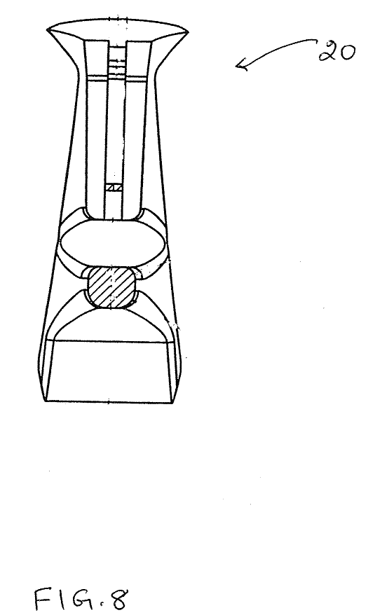

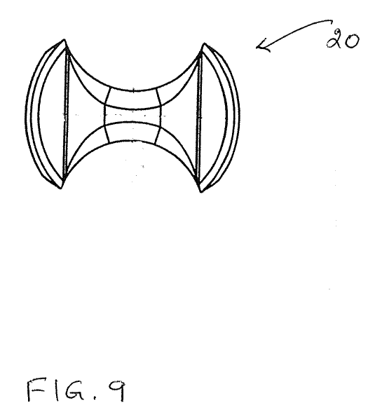

Referring now to the drawings, where like elements are designated by like reference numerals, FIGS. 1-10 illustrate exemplary embodiments of a harness/bolt graft assembly 100 for supporting a ligament graft in a bone tunnel. As shown in FIGS. 1-9, the harness/bolt graft assembly 100 comprises a graft harness 20 that securely engages a bolt 50.

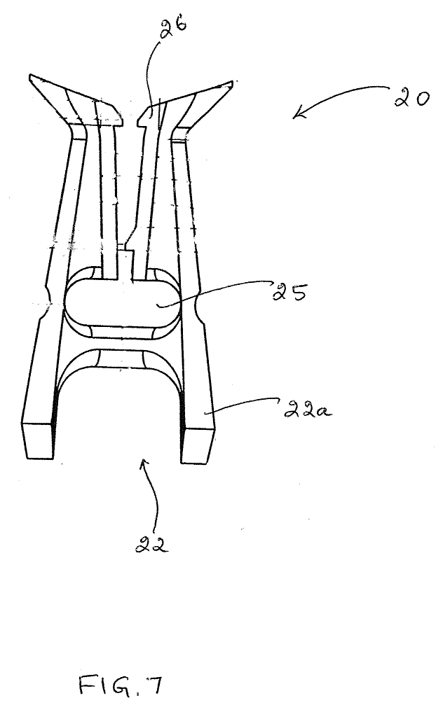

Harness 20 preferably takes the form illustrated in FIG. 1 and may be made from a biocompatible material, and preferably from bioabsorbable materials, bone, or synthetic bone. As illustrated in FIGS. 1 and 7, harness 20 is provided at its distal end 21 with a radial groove 22, which accepts a looped ligament graft and which is defined by symmetrical lateral sides 22a.

According to an exemplary embodiment of the present invention, the harness 20 may be provided with a suture passageway 25 through its body, to allow a ligament graft to be subsequently secured (by suturing, for example) to the harness.

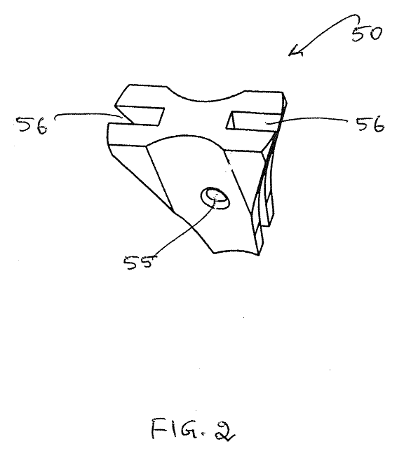

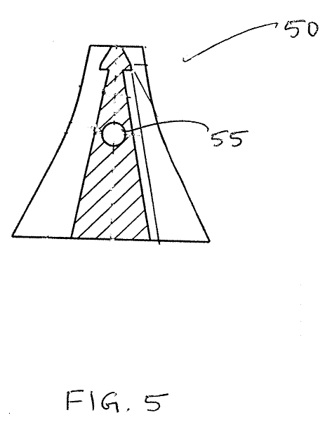

As illustrated in FIGS. 2 and 5, the bolt 50 is provided with a transverse through-hole 55, preferably perpendicular to the radial groove 22 of the harness 20, to accommodate a suture strand having a size up to #5 in diameter. Passageway 55, shown as a through-hole in FIGS. 2 and 5, also can be formed as a slot to accommodate additional sutures and sutures of larger size. The suture strand passed through passageway 55 is used to draw the harness/graft assembly into a bone tunnel, as explained below.

The bolt 50 is also provided with side cannulations 56 which are configured to engage corresponding protuberances 26 provided on the inner proximal sides of the harness 20, to allow the bolt 50 to engage and lock on to the harness 20 after the harness has been inserted in the femoral tunnel. Inserting the bolt 50 into the harness 20 causes the legs of the harness to separate, thereby engaging the tunnel wall.

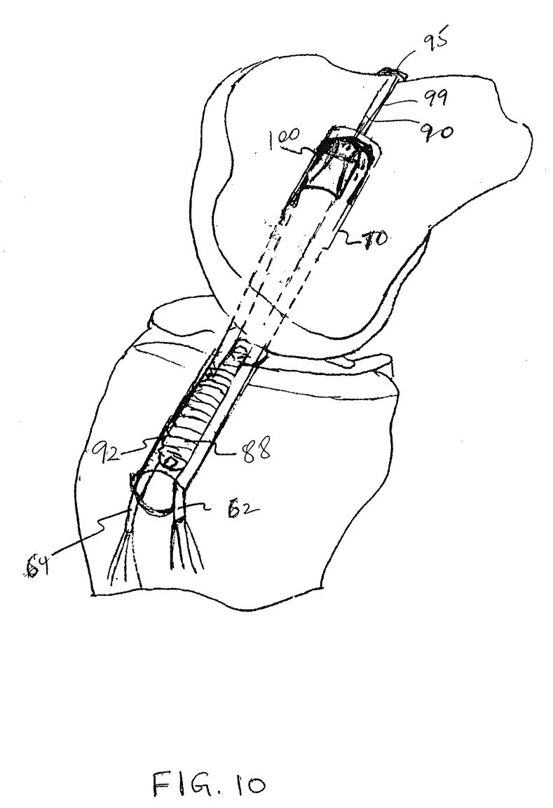

Soft tissue graft 70 (FIG. 10) is preferably looped and draped over the harness 20 of the harness/bolt assembly 100 of the present invention, and then may be securely attached to the harness by suturing, for example, or a combination of suturing and additional fixation techniques. In this manner, slippage of the graft out of the groove 22 is virtually eliminated and the orientation of the double bundle graft in the femoral opening is predetermined and fixed. If desired, a suture strand, or a plurality of suture strands, with corresponding tied suture knots, may be employed to securely fix the graft ligament to the harness.

FIG. 10 illustrates the harness/graft assembly 100 fully inserted and seated in femoral tunnel 90. A preferred method of ligament fixation according to the present invention involves preparing a femoral tunnel 90 and a tibial tunnel 92. Prior to installation, soft tissue graft 70 is looped and draped over the harness 20 and preferably securely attached to the harness 20 by sutures, to form the harness/bolt graft assembly 100. The legs 62, 64 of the graft 70 are combined to form a double bundle which approximates the anatomical orientation of the original tendon, and which correspondingly approximates the double bundle biomechanical function of the double bundle native ligament, whereby one ligament strand tensions in knee extension and the other ligament strand tensions in knee flexion.

The prepared double-bundle graft supported by the harness is passed through the femoral tunnel 90 by pulling on suture 99 which is passed through hole 55 of the bolt 50. The bolt 50 is then pushed forward into the harness 20, spreading the legs of the harness to engage the wall of the femoral tunnel. To complete the femoral fixation, suture 99 may be subsequently affixed femur after exiting the femoral tunnel 90 using a button 95, for example, a staple, or any other fixation device.

Tibial tunnel fixation can be accomplished using methods disclosed in co-pending U.S. Patent Application Publication No. 2005/0096743, entitled METHOD FOR CREATING A DOUBLE BUNDLE LIGAMENT ORIENTATION IN A SINGLE BONE TUNNEL DURING KNEE LIGAMENT RECONSTRUCTION, of common assignment with the present application, and incorporated herein by reference herein in its entirety. The fixation method described in U.S. Patent Application Publication No. 2005/0096743 relates to the use of a notcher, burr, or rasp to widen the tunnels 90, 92 into a substantially oval shape. The tunnel shape attained is sufficiently wide to accommodate a fixation implant between the two strands of the replacement graft inserted into the tunnels.

The methods and devices of the present invention can be applied to anterior or posterior cruciate ligament reconstruction in the knee, for example. The methods and devices also can be adapted easily by one of skill in the art for ligament reconstruction in a variety of joints, including those in humans and otherwise.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Additions, deletions, substitutions, and other modifications can be made without departing from the spirit or scope of the present invention.

I claim:

1. A graft harness assembly comprising:
a harness for support a ligament graft in a bone tunnel, comprising a proximal end formed of a pair of spreadable legs, and a distal end having a groove for supporting a loop of the graft; and
a bolt adapted to be received in the proximal end of the harness for spreading the legs of the harness to engage the graft harness assembly to the bone tunnel.

2. A graft assembly according to claim 1, wherein the proximal end of the harness has an opening to receiving the bolt, the opening having a surface with protuberances configured to securely engage the bolt upon insertion thereof.

3. A graft assembly according to claim 1, wherein the bolt further comprising a transverse hole for receiving a flexible member which is used to draw and fixate the graft harness assembly into a bone tunnel.

4. A graft assembly according to claim 1, wherein at least a portion of the ligament graft is secured to the harness by suture.

5. A graft assembly according to claim 1, wherein the harness is formed of a bioabsorbable material.

6. A method of knee reconstruction using a graft harness assembly comprising:
forming a tibial tunnel and a femoral tunnel, each tunnel having tunnel walls;
looping a ligament graft over a harness, the harness having spreadable legs;
inserting the graft harness assembly, with the graft looped over the harness, into the femoral tunnel; and
securing the graft harness assembly in the femoral tunnel by advancing a bolt into the harness, spreading the legs of the harness to engage the wall of the femoral tunnel.

7. A method according to claim 7, wherein the step of inserting the graft harness assembly in the femoral tunnel includes the step of pulling the graft harness assembly with the looped graft through the tibial tunnel and into the femoral tunnel using a suture strand passed through a transverse hole in the bolt.

8. A method according to claim 8, further comprising the step of affixing the suture strand to the femur to further secure the graft harness assembly in the femoral tunnel.

9. A method according to claim 9, wherein the ligament graft comprises at least two graft strands and the ligament graft is secured in the tibial tunnel by installing a respective implant between two strands of the graft such that the graft strands are secured separately between an outer wall of the respective implant and a tibial tunnel wall.

10. A method according to claim 10, wherein the step of securing the ligament graft in the tibial tunnel includes securing the graft in the tibial tunnel using an interference screw implant.
